(19) United States
(12) Patent Application Publication
(10) Pub. No.: US 2020/0375733 A1
(43) Pub. Date: Dec. 3, 2020

(54) DEVICES, SYSTEMS AND METHODS FOR COLLAPSIBLE AND EXPANDABLE IMPLANT LOADING, TRANSSEPTAL DELIVERY, POSITIONING DEPLOYMENT AND REPOSITIONING DEPLOYMENT

(71) Applicant: 4C Medical Technologies, Inc., Maple Grove, MN (US)

(72) Inventors: Jason S. Diedering, Minneapolis, MN (US); Sounthara Khouengboua, Maple Grove, MN (US); Saravana B. Kumar, Minnetonka, MN (US)

(21) Appl. No.: 16/877,887

(22) Filed: May 19, 2020

Related U.S. Application Data
(60) Provisional application No. 62/854,584, filed on May 30, 2019.

(51) Int. Cl. A61F 2/24

(52) U.S. Cl. CPC A61F 2/2427; A61F 2/2418

(57) ABSTRACT

A loading, delivery, deployment and positioning system for a prosthetic heart valve device includes a stent that is biased to expand and adapted to collapse into the lumen of a delivery catheter. A torque wire of the system includes a distal threaded region, has length that is longer than a length of the delivery catheter, and is adapted for movement within the delivery catheter lumen. A stent cap is non-rotationally attached at or near the top of the stent and defines a channel and a pair of lateral locking grooves therethrough. A male engagement member of the system includes a threaded region adapted to threadingly engage the threaded region of the torque wire, a stem region extending distally from the threaded region; and engagement handles extending laterally from a distal end of the stem region, the engagement handles being adapted to detachably engage the stent cap.

### CROSS-REFERENCE TO RELATED APPLICATIONS

This application claims the benefit of U.S. Provisional Application No. 62/854,584, filed May 30, 2019 and entitled DEVICES, SYSTEMS AND METHODS FOR COLLAPSIBLE AND EXPANDABLE IMPLANT LOADING, TRANSSEPTAL DELIVERY, POSITIONING DEPLOYMENT AND REPOSITIONING DEPLOYMENT, the entirety of which is hereby incorporated by reference.

### STATEMENT REGARDING FEDERALLY SPONSORED RESEARCH OR DEVELOPMENT

Not Applicable

### BACKGROUND OF THE INVENTION

### Field of the Invention

The invention relates to devices, systems and features for loading, delivering, positioning and repositioning a stent in a body. More specifically, an expandable and collapsible prosthetic heart valve device is delivered and positioned within a heart chamber, preferably transseptally to the left atrium.

### Description of the Related Art

The human heart comprises four chambers and four heart valves that assist in the forward (antegrade) flow of blood through the heart. The chambers include the left atrium, left ventricle, right atrium and right ventricle. The four heart valves include the mitral valve, the tricuspid valve, the aortic valve and the pulmonary valve. See generally FIG. 1.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood into the chamber it just flowed out of. For example, the mitral valve when working properly provides a one-way valving between the left atrium and the left ventricle, opening to allow antegrade flow from the left atrium to the left ventricle and closing to prevent retrograde flow from the left ventricle into the left atrium. This retrograde flow, when present, is known as mitral regurgitation or mitral valve regurgitation.

FIG. 2 illustrates the relationship between the left atrium, annulus, chordae tendineae and the left ventricle relative to the mitral valve leaflets. As is shown, the upper surface of the annulus forms at least a portion of the floor or lower surface of the left atrial chamber, so that for purposes of description herein, the upper surface of the annulus is defined as marking the lower boundary of the left atrial chamber.

Native heart valves may be, or become, dysfunctional for a variety of reasons and/or conditions including but not limited to disease, trauma, congenital malformations, and aging. These types of conditions may cause the valve structure to fail to close properly resulting in regurgitant retrograde flow of blood from the left ventricle to the left atrium in the case of a mitral valve failure. FIG. 3 illustrates regurgitant blood flow with an exemplary dysfunctional mitral valve.

Mitral valve regurgitation is a specific problem resulting from a dysfunctional mitral valve that allows at least some retrograde blood flow back into the left atrium from the right atrium. In some cases, the dysfunction results from mitral valve leaflet(s) that prolapse up into the left atrial chamber, i.e., above the upper surface of the annulus instead of connecting or coapting to block retrograde flow. This backflow of blood places a burden on the left ventricle with a volume load that may lead to a series of left ventricular compensatory adaptations and adjustments, including remodeling of the ventricular chamber size and shape, that vary considerably during the prolonged clinical course of mitral regurgitation.

Regurgitation can be a problem with native heart valves generally, including tricuspid, aortic and pulmonary valves as well as mitral valves.

Native heart valves generally, e.g., mitral valves, therefore, may require functional repair and/or assistance, including a partial or complete replacement. Such intervention may take several forms including open heart surgery and open heart implantation of a replacement heart valve. See e.g., U.S. Pat. No. 4,106,129 (Carpentier), for a procedure that is highly invasive, fraught with patient risks, and requiring not only an extended hospitalization but also a highly painful recovery period.

Less invasive methods and devices for replacing a dysfunctional heart valve are also known and involve percutaneous access and catheter-facilitated delivery of the replacement valve. Most of these solutions involve a replacement heart valve attached to a structural support such as a stent, commonly known in the art, or other form of wire network designed to expand upon release from a delivery catheter. See, e.g., U.S. Pat. No. 3,657,744 (Ersek); U.S. Pat. No. 5,411,552 (Andersen). The self-expansion variants of the supporting stent assist in positioning the valve, and holding the expanded device in position, within the subject heart chamber or vessel. This self-expanded form also presents problems when, as is often the case, the device is not properly positioned in the first positioning attempt and, therefore, must be recaptured and positionally adjusted. This recapturing process in the case of a fully, or even partially, expanded device requires re-collapsing the device to a point that allows the operator to retract the collapsed device back into a delivery sheath or catheter, adjust the inbound position for the device and then re-expand to the proper position by redeploying the positionally-adjusted device distally out of the delivery sheath or catheter. Collapsing the already expanded device is difficult because the expanded stent or wire network is generally designed to achieve the expanded state which also resists contractive or collapsing forces.

Besides the open heart surgical approach discussed above, gaining access to the valve of interest is achieved percutaneously via one of at least the following known access routes: transapical; transfemoral; transatrial; and trans septal delivery techniques.

Generally, the art is focused on systems and methods that, using one of the above-described known access routes, allow a partial delivery of the collapsed valve device, wherein one end of the device is released from a delivery sheath or catheter and expanded for an initial positioning followed by full release and expansion when proper positioning is achieved. See, e.g., U.S. Pat. No. 8,852,271 (Murray, III); U.S. Pat. No. 8,747,459 (Nguyen); U.S. Pat. No. 8,814,931 (Wang); U.S. Pat. No. 9,402,720 (Richter); U.S. Pat. No. 8,986,372 (Murray, III); and U.S. Pat. No. 9,277,991 (Salahieh); and U.S. Pat. Pub. Nos. 2015/0272731 (Racchini); and 2016/0235531 (Ciobanu).

In addition, all known prosthetic heart valves are intended for full replacement of the native heart valve. Therefore, these replacement heart valves, and/or anchoring or tethering structures, physically extend out of the left atrial chamber, in the case of mitral valves, and engage the inner annulus and/or valve leaflets, in many cases pinning the native leaflets against the walls of the inner annulus, thereby permanently eliminating all remaining functionality of the native valve and making the patient completely reliant on the replacement valve. In other cases, the anchoring structures extend into the left ventricle and may anchor into the left ventricle wall tissue and/or the sub-annular surface at the top of the left ventricle. Others may comprise a presence in, or engagement with, a pulmonary artery.

Obviously, there will be cases when native valve has lost virtually complete functionality before the interventional implantation procedure. In this case the preferred solution will comprise an implant that does not extent outside of, e.g., the left atrium, and that functions to completely replace the native valve function. However, in many other cases, the native valve remains functional to an extent and may, or may not, continue to lose functionality after the implantation procedure. A preferred solution in this case comprises delivery and implantation of a valve device that will function both as a supplemental or augmentation valve without damaging the native leaflets in order to retain native valve leaflet functionality as long as present, while also being fully capable of replacing the native function of a valve that slowly loses most or all of its functionality post-implantation of the prosthetic valve.

Delivery systems, devices and methods for prosthetic heart valve devices are known, but require improvement. In particular, known transseptal delivery systems, devices and methods can be improved on, including but not limited to: the collapsing/loading of the prosthetic heart valve device into the lumen of a delivery catheter; the release and orientation of the expanding prosthetic heart valve device from the distal end of the delivery catheter's lumen into the heart chamber, and oriented positioning within the heart chamber. Known delivery systems, devices, and methods also still suffer from significant flaws in delivery methodology including, inter alia, recapture capability and efficiency to enable repositioning as needed to achieve optimal locating and sealing.

Various embodiments of the several inventions disclosed herein address these, inter alia, issues.

### BRIEF SUMMARY OF THE INVENTION

The present invention provides methods, devices, and systems for improved collapsing/loading of a prosthetic heart valve device into a lumen of a delivery catheter; improved release and orientation of the expanding prosthetic heart valve device from the distal end of the delivery catheter's lumen into a heart chamber, and improvements in oriented positioning of the device within the heart chamber. The methods, devices, and systems of the present disclosure also provide improved capability and efficiency of recapturing a delivered prosthetic heart valve device to enable repositioning, as needed, to achieve optimal locating and sealing of the device at the desired treatment site. These improvements may be at least partially achieved by a stent cap affixed to a top portion of a stent of a prosthetic valve device and configured for engagement and disengagement with a male engagement member, the male engagement member in turn being configured for engagement and disengagement with a manipulable torque wire that enables positioning, release, recapture, and repositioning of the prosthetic valve device via the male engagement member and the stent cap.

In one embodiment, a loading, delivery, deployment and positioning system for an expandable and collapsible prosthetic heart valve device having a stent outer frame with a top and a bottom, wherein the bottom defines an outflow region therefrom, the prosthetic heart valve device biased to expand and adapted to collapse into the lumen of a delivery catheter, comprises a torque wire having a length that is longer than a length of the delivery catheter and adapted to translate and/or rotate within the delivery catheter lumen when a distal end of the torque wire is manipulated by an operator and wherein the torque wire comprises a threaded region at its distal end; a stent cap non-rotationally attached at or near the top of the stent outer frame, the stent cap defining a channel and a pair of lateral locking grooves therethrough, wherein the channel is defined continuously with the lateral locking grooves; a male engagement member comprising: a threaded region at a proximal end, the threaded region adapted to threadingly engage the threaded region of the torque wire, a stem region extending distally from the threaded region; and left and right engagement handles extending laterally from a distal end of the stem region, wherein the left and right engagement handles are adapted to detachably engage the stent cap.

In another embodiment, a method for loading, delivery, deployment and positioning a system for an expandable and collapsible prosthetic heart valve device comprises providing the system of the one embodiment described above; threadingly attaching the male engagement member to the torque wire; removably attaching the male engagement member to the stent cap; pulling the torque wire through the lumen of the delivery catheter in a proximal direction; pulling the expanded prosthetic heart valve device into the distal end of the delivery catheter lumen and thereby collapsing the prosthetic heart valve device therein; locating and loading the collapsed prosthetic heart valve device within the delivery catheter lumen; accessing the patient's heart chamber with the distal end of the delivery catheter; pushing with the torque wire the collapsed prosthetic heart valve device out of the distal end of the delivery catheter, whereby the prosthetic heart valve device biasingly expands; rotating and/or otherwise turning the torque wire to direct and position the expanding prosthetic heart valve device within the heart chamber; disconnecting the male engagement member from the stent cap; withdrawing the torque wire and attached male engagement member into the lumen of the delivery catheter; and withdrawing the delivery catheter from the patient's body.

Certain inventive embodiments described herein are readily applicable to single or two chamber solutions, unless otherwise indicated. Moreover, certain embodiments discussed herein may be applied to preservation and/or replacement of native valve functionality generally, and are not, therefore, limited to prosthetic mitral valve devices but may be extended to include prosthetic tricuspid valve devices, prosthetic aortic devices, prosthetic pulmonary valves, and methods for the loading, delivery, deployment, and positioning of any such valves.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

### BRIEF DESCRIPTION OF THE SEVERAL VIEWS OF THE DRAWINGS

FIG. 1 illustrates certain features of the heart in cross-section.

FIG. 2 illustrates a cross-sectional perspective view of the left side of the heart.

FIG. 3 illustrates a cross-sectional view of the heart showing retrograde blood flow resulting from mitral valve regurgitation compared with normal blood flow.

FIG. 4 illustrates a partial cutaway side view of a prosthetic heart valve device of one embodiment of the present invention.

FIG. 5 illustrates a perspective view of a stent cap of one embodiment of the present invention.

FIG. 6 illustrates a perspective view of a male engagement member of embodiment of the present invention.

FIG. 7 illustrates a perspective view of a stent cap of attached to a stent of an exemplary prosthetic heart valve device in one embodiment of the present invention.

FIG. 8A illustrates a perspective view of a male engagement member connected to a stent cap and a torque wire in one embodiment of the present invention.

FIG. 8B illustrates a side partial cutaway view of a male engagement member connected to a torque wire and partially received within a delivery catheter in one embodiment of the present invention.

FIGS. 9A-9L illustrate exemplary method steps for an exemplary transseptal delivery and positioning of a prosthetic heart valve device using an embodiment of the present invention.

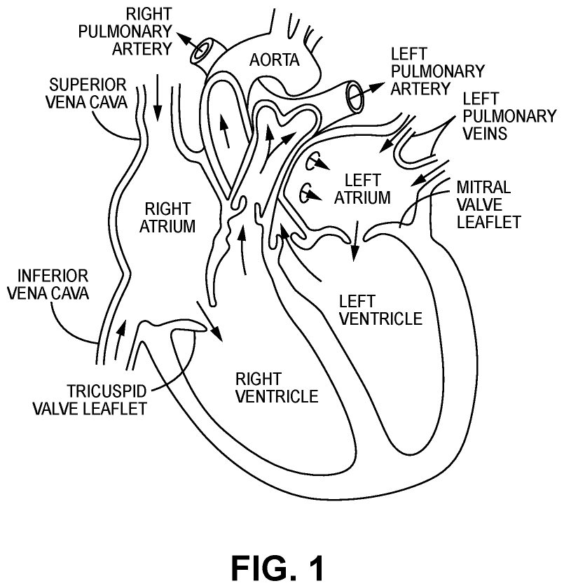

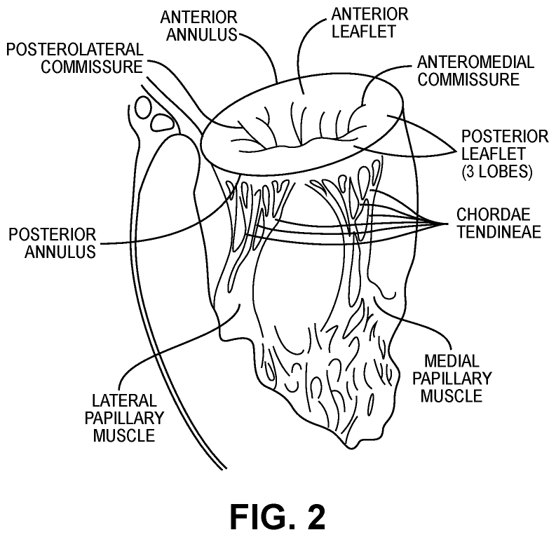

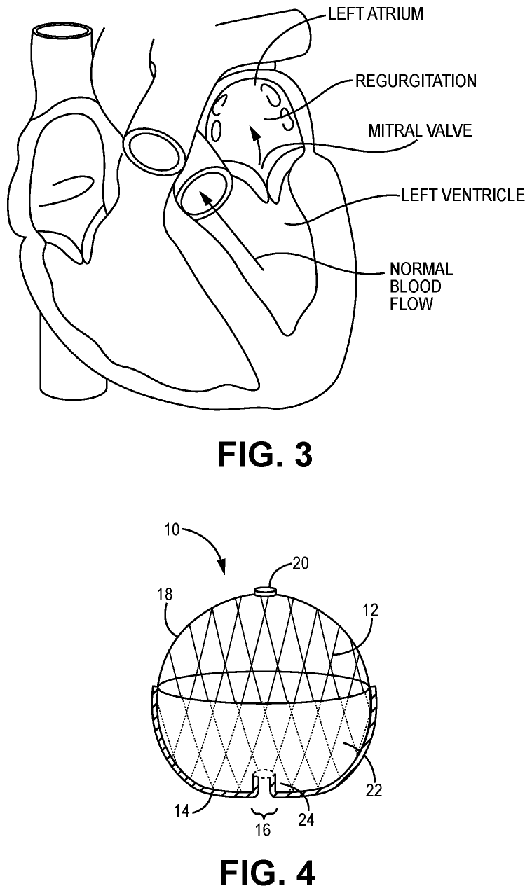

### DETAILED DESCRIPTION OF THE INVENTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

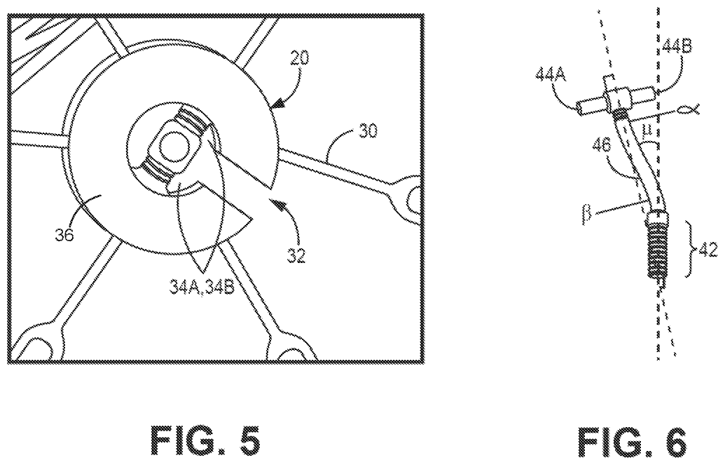

FIGS. 4-9L illustrate various embodiments of devices of the present invention and methods of their use. Though these embodiments are illustrated and described separately, it will be understood by the skilled artisan that aspects of one or more of the embodiments may be combined.

Generally, various embodiments of the present invention are directed to devices and methods for optimizing delivery of a prosthetic heart valve device comprising a collapsible and expandable frame, e.g., a stent or other collapsible and expandable device. The embodiments described herein optimize delivery of a prosthetic heart valve device by (1) reducing loading forces during collapsing and translating through the delivery catheter lumen; and/or (2) by reducing, minimizing or eliminating air introduction into the system comprising the prosthetic heart valve device and/or the lumen of the delivery catheter. The embodiments described herein also provide improved capability and efficiency of recapturing a delivered prosthetic heart valve device to enable repositioning, as needed, to achieve optimal locating and sealing of the device at the desired treatment site.

FIG. 4 illustrates a side view of a prosthetic heart valve device 10 of one embodiment of the present invention. The prosthetic heart valve device 10 includes a stent outer frame 12 defining a bottom portion 14 having an outflow region 16. The stent 12 further includes a top portion 18 and a female stent cap 20 affixed to top portion 18 at the top outer end of the stent 12. In the embodiment of FIG. 4, the exemplary stent 12 comprises a ball-like shape, though other shapes are within the scope of the present invention and may include similar features, such as an outflow region and a top portion to which a female stent cap (e.g., stent cap 20) may be affixed.

The device 10 further includes a valve support 24 that contains prosthetic leaflet(s) (not shown) and provides a flow channel for blood flow through the stent 12 to the outflow region 16. When implanted, the valve support 24 is adapted to substantially align with an annulus and allow one-way, antegrade blood flow therethrough while preventing retrograde blood flow as a result of the prosthetic leaflet(s) supported therein.

The valve support 24 may be wholly contained within the interior of the stent 12 or may extend at least partially out of the stent 12 in the downstream (outflow) direction. Still more alternatively, the valve support 24 may extend completely outwardly from the stent 12, and not extend radially into the interior of the stent 12. As shown in FIG. 4, the bottom portion 14 of the stent 12 may be at least partially covered with a skirt 22, shown surrounding or covering the exterior of a portion of the bottom portion 14 of stent 12's frame. The skirt 22 may be formed of a material that conforms and seals with portions of the atrial wall and/or upper annular surface. In embodiments in which the valve support 24 extends outwardly from the stent 12 and below the annular surface when implanted, at least a portion of the valve support 24 may be covered with material of the skirt 22. A skirt formed of such a material may also, or in the alternative, cover the interior of a portion of the frame of stent 12.

The stent cap 20 preferably may be affixed to the stent 12 at the midline or longitudinal axis of the prosthetic heart valve device 10, though other locations proximate the top portion 18 of the frame of stent 12 are also possible and within the inventive scope of the present disclosure.

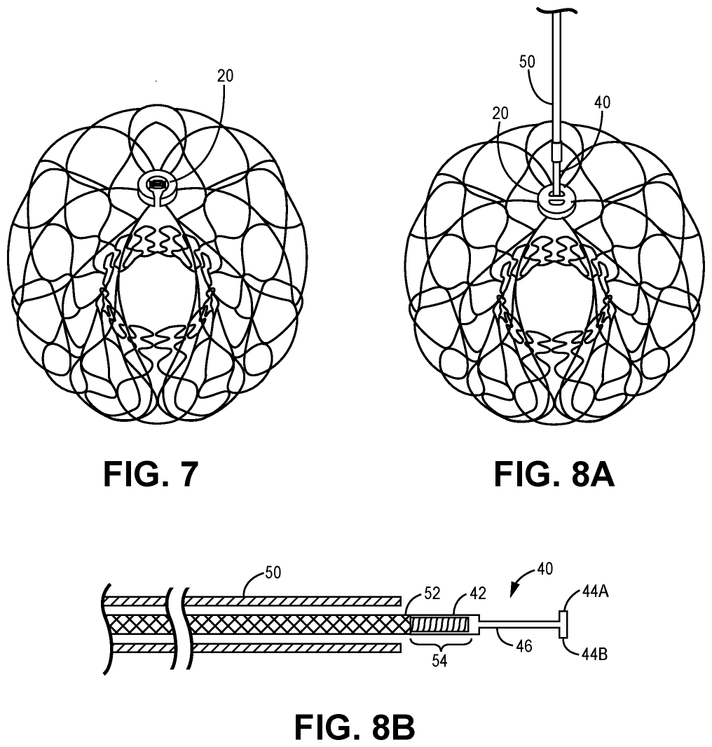

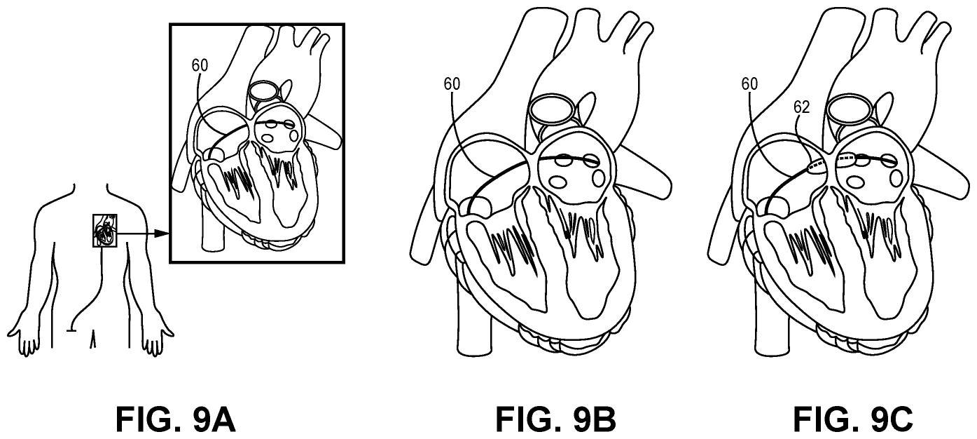

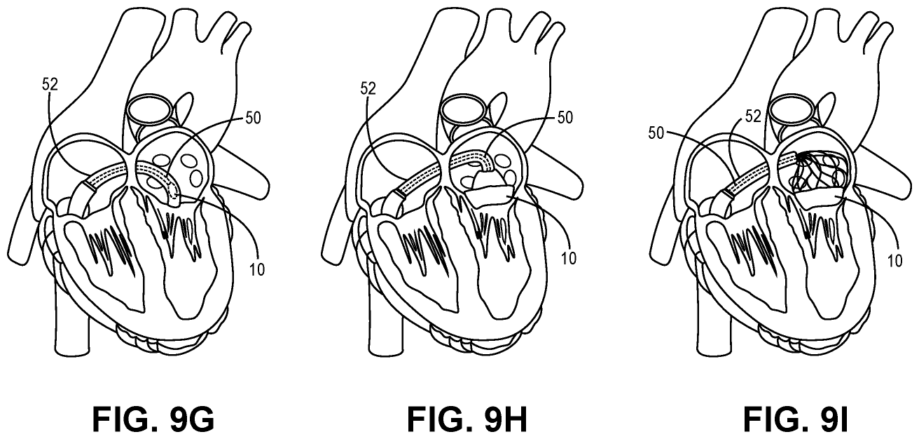

FIG. 5 illustrates a perspective view of a stent cap of one embodiment of the present invention; e.g., the stent cap 20 of the prosthetic heart valve device 10 illustrated in FIG. 4. In the embodiment of FIG. 5, the stent cap 20 is generally flat on its top surface, though other shapes and surface profiles of a stent cap may be provided. When the prosthetic heart valve device 10 is expanded and positioned, the top surface of the stent cap 20 is pressed against the upper surface or roof of the subject heart chamber, e.g., the left atrium. The stent cap 20 has a female configuration that enables detachable engagement of the stent cap 20 with a male engagement member (discussed below with respect to FIGS. 6 and 8A) that in turn is configured to engage a torque wire to allow for loading transseptal deployment, recapture, and repositioning of the prosthetic heart valve device 10.

As shown in FIG. 5, the stent 12 comprises a plurality of struts 30. The stent cap 20 comprises a fixed, non-rotational connection to more than one strut 30 of the stent 12. The fixed, non-rotational connection of the stent cap 20 to struts 30 of the stent 12 may be achieved by any suitable manner, including but not limited to welding, soldering, an interference fit between the struts 30 and a corresponding plurality of grooves formed in a bottom side of the stent cap 20, or any other suitable manner. In some embodiments, the stent cap 20 may be formed from titanium, a titanium alloy, or other suitable material.

The stent cap 20 comprises a cap body 36 that defines an access channel 32 therethrough. When the stent cap 20 is affixed to the stent 12, the access channel 32 is spaced apart from struts 30 of the stent 12 to enable unimpeded access of a male engagement member to the channel 32. The access channel 32 merges into lateral locking grooves 34A and 34B, which are also defined by the cap body 36. The lateral locking grooves 34A and 34B comprise a radial (the largest) diameter that is larger than the radial diameter of the access channel 32. In another embodiment, the center of the stent cap 20 may include a female threaded attachment that allows for engagement of a male threaded component of a male engagement member thereto. As with other embodiments, embodiment in which the center of the stent cap 20 includes a female threaded attachment advantageously may enable loading of a prosthetic heart valve device (e.g. device 10) into a delivery catheter, transseptal deployment of the prosthetic heart valve device, and repositioning of the prosthetic heart valve device as needed.

FIG. 6 illustrates a perspective view of one embodiment of a male engagement member 40 that is designed to detachably engage the stent cap 20. The male engagement member 40 defines a threaded region 42 at its proximal end, a handle region 44 at its distal end, and a stem region 46 extending between the threaded region 42 and the handle region 44. The handle region 44 defines a left engagement handle 44A and a right engagement handle 44B, each of which extend laterally a distance away from the stem region 46. The stem region 46 extends proximally away from the left and right engagement handles, terminating at the proximal end in a series of threads. The right and left engagement handles 44A, 44B may be dimensioned such that a combined length of the engagement handles 44A, 44B is longer than a combined length of the lateral locking grooves 34A, 34B of the stent cap 20. In this manner, when the stem region 46 is introduced into the channel 32 of the stent cap 20 and the male engagement member 40 is manipulated to advance the engagement handles 44A, 44B toward the lateral locking grooves 34A, 34B, the engagement handles 44A, 44B are retained within the stent 12 below the lateral locking grooves 34A, 34B such that the male engagement member 40 is releasably engaged with the stent cap 20. As further discussed below with respect to FIG. 8A, the stem region 46 of the male engagement member 40 may be withdrawn through the channel 32 and out of the stent 12 to release of the stent cap 20 from the male engagement member 40.

In embodiments in which the center of the stent cap 20 includes a female threaded attachment, the male engagement member 40 may include a second male threaded region at the distal end of the male engagement member 40. In some such embodiments, the threaded region 42 at the proximal end of the male engagement member 40 and the second male threaded region at the distal end of the male engagement member 40 may be threaded in opposite directions (i.e. one having right-hand threading and the other having left-hand threading). In this manner the male engagement member 40 may be unscrewed from the female threaded attachment of the stent cap 20 to release of the stent cap 20 from the male engagement member 40 without unscrewing the threaded region 42 from a torque wire (described with respect to FIGS. 8A and 8B).

As further shown in the embodiment of FIG. 6, the stem region 46 of the male engagement member 40 comprises a curvilinear shape, though in other embodiments, the stem region 46 may comprise a straight or linear shape. In embodiments in which the stem region 46 is curvilinear, the stem may comprise a single curve or a radius. Alternatively, as illustrated, stem region 46 may comprise more than one curve, e.g., a distal curve comprising a radius or degree of curvature α and a more proximal curve of a radius or degree of curvature β. The degree of curvature α may be measured relative to a line drawn from the center of the left and right engagement handles 44A, 44B and perpendicular thereto where the stem region 46 meets the handle region 44, as shown in FIG. 6. The degree of curvature β may be measured relative to this line where the stem region 46 meets the threaded region 42, as further shown in FIG. 6. The curvatures for distal and proximal curves, respectively α and β, may comprise substantially equal curvatures or may be different from each other.

Moreover, the line drawn from the center of the left and right engagement handles 44A, 44B and perpendicular thereto, as shown, may be non-parallel to, and therefore intersecting with, a line running through the central axis of the threaded region 42, with a predetermined angle of offset, represented as μ. Alternatively, the perpendicular line to the left and right engagement handles 44A, 44B and the line through the central axis of the threaded region 42 may be parallel with each other. Still more alternatively, the perpendicular line, drawn in the center of the handle region 44 between the left and right engagement handles 44A, 44B, may be collinear with the line through the central axis of the threaded region 42. In any such embodiments, the various curvatures of the stem region 46 of the male engagement member 40 advantageously may assist or enable downward turning and/or other directional manipulation of the prosthetic heart valve device 10 during delivery of the device 10.

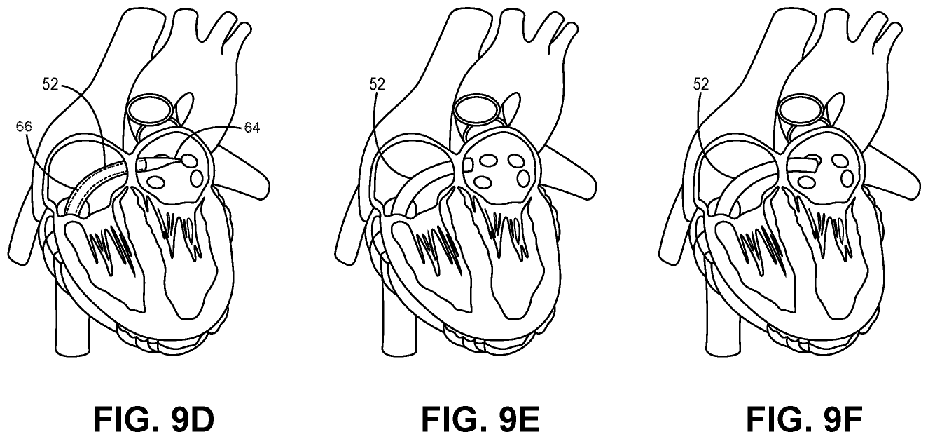

FIG. 7 illustrates a perspective view of the stent cap 20 attached at the top portion 18 of the stent 12, without the male engagement member attached thereto. The channel 32 and lateral locking grooves 34A, 34B can be seen in FIG. 7, with the stent cap 20 affixed to the stent 12 such that the channel 32 is aligned with a space between two struts 30 at the top portion 18. By positioning the stent cap 20 in this manner relative to the stent 12, access to the channel 32 is not impeded by the struts 30.

FIGS. 8A and 8B illustrate the connection of the male engagement member to a torque wire that may be rotationally and translationally engaged in the lumen of the delivery catheter, in one embodiment of the present invention. FIG. 8A illustrates a perspective view of male engagement member 40 connected to stent cap 20 and a torque wire 50, the torque wire 50 engaged within a lumen of a delivery catheter 52. As discussed above, the male engagement member 40 may be connected to the stent cap 20 by introducing the stem region 46 of the male engagement member 40 into the channel 32 of the stent cap 20 and advancing the male engagement member 40 such that the engagement handles 44A, 44B are retained by the lateral locking grooves 34A, 34B. This may be done prior to loading the prosthetic heart valve device 10 into the delivery catheter 52 for delivery to the treatment site.

As shown in FIG. 8A, the stem region 46 of the male engagement member 40 has been translated through the access channel 32 of the stent cap 20, with the left and right engagement handles 44A, 44B disposed below the stent cap 20; i.e., within the interior of the body or frame of stent 12. The left and right engagement handles 44A, 44B comprise a length that is longer than the lateral locking grooves 34A, 34B of the stent cap 20, so that when the male engagement member 40 is positioned therein, the male engagement member 40 and stent cap 20, and therefore the body of stent 12 and entire prosthetic heart valve device 10, will rotate together. The male engagement member 40 may be adapted to rotate or pivot, in response to, inter alia, a rotation of the attached torque wire 50, in the access channel 32 of the stent cap 20. Male engagement member 40 may also translate through the access channel 32 of the stent cap 20 so that the relationship and/or position and/or orientation of the male engagement member 40 may change relative to the stent cap 20. When an operator desires to release the male engagement member 40 from the stent cap 20, this may be accomplished by manipulating the torque wire 50 to withdraw the stem region 46 of the male engagement member 40 back through the access channel 32 until the left and right engagement handles 44A, 44B clear the entrance to the channel 32, and then withdrawing the male engagement member 40 out from the interior of the stent 12.

FIG. 8B illustrates a side partial cutaway view of the male engagement member connected to the torque wire 50 and partially received within a lumen defined by the delivery catheter 52. The torque wire 50 comprises a complementary threading structure 54 adapted to threadingly engage the threaded region 42 of the male engagement member 40. Rotation of the torque wire 50 either threads or unthreads the male engagement member 40 to, or from, the complementary threading structure 54 of the torque wire. When threadingly engaged to the torque wire 50, the male engagement member 40 may be translated and rotated by an operator on the proximal end of the delivery catheter 52 and the torque wire 50. As shown in FIG. 8B, the complementary threading structure 54 of the torque wire 50 may be threadingly engaged with the threaded region 42 of the male engagement member 40 when the proximal portion of the torque wire 50 is disposed in the lumen of the delivery catheter 52.

Applicant has found that the torque wire 50 provides necessary tensile strength for not only pushing and pulling of the prosthetic heart valve device 10, but also for translatable rotation of the prosthetic heart valve device 10 initiated from the proximal handle end of the torque wire 50 to optimize positioning of the prosthetic heart valve device 10 within the heart chamber. Once the prosthetic heart valve device 10 is connected with the torque wire 50 in this manner, the expanded stent 12 may be collapsingly loaded into the lumen of the delivery catheter 52 by retracting or pulling the torque wire 50 distally. Similarly, after expanded delivery of the prosthetic heart valve device 10 into the heart chamber, the prosthetic heart valve device 10 may be resheathed into the lumen of the delivery catheter 52 by pulling the torque wire 50 distally. The stent cap 20 may be translated when the bottom portion 14 of the body of the stent 12, i.e., the portion comprising the valve support 24, is engaged with the anatomy of the patient at the treatment site.

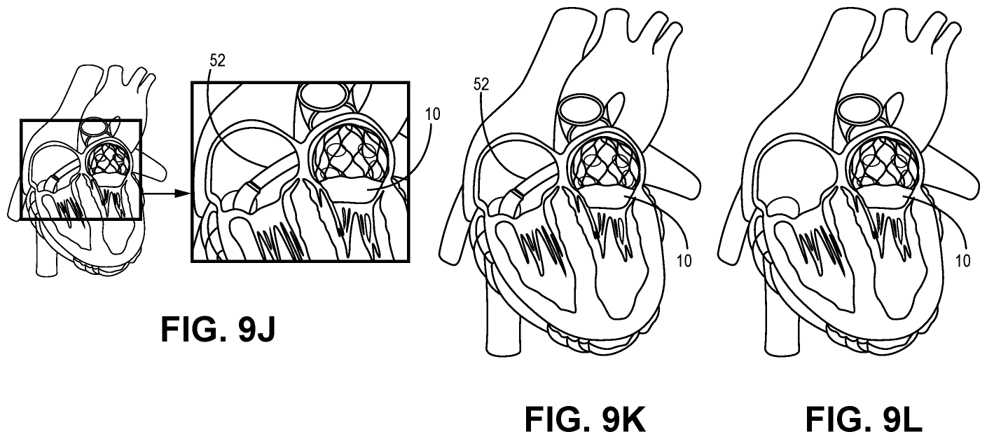

FIGS. 9A-9L illustrate exemplary method steps for an exemplary transseptal delivery and positioning of a prosthetic heart valve device; e.g., the prosthetic heart valve device 10, using an embodiment of the present invention. Initially, the torque wire 50 is connected with the male engagement member 40 as described above with respect to FIGS. 8A and 8B. The male engagement member 40 is, in turn, connected with the stent cap 20 of the prosthetic heart valve device 10 also as described above. Next, the biasingly expanded frame of the stent 12 of the prosthetic heart valve device 10 is loaded into the lumen of the delivery catheter 52 by pulling the torque wire 50 in a proximal direction, thereby collapsing the frame of the stent 12 into the lumen of the delivery catheter 52. When the prosthetic heart valve device 10 is properly positioned within the lumen of the delivery catheter 52 in this manner, the prosthetic heart valve device 10 is consider “loaded” in the delivery catheter 52 in a collapsed position.

FIGS. 9A and 9B illustrate a guide wire 60, which has been advanced through the septum between the right and left atria of the patient, using femoral access. In some embodiments, the guide wire 60 may include an expandable member, e.g., a balloon 62, disposed thereon and may define an inflation lumen defining one or more apertures within the balloon 62, as is well-known in the art. With the balloon 62 positioned as illustrated in FIG. 9C, a fluid may be delivered to the balloon 62 via the inflation lumen of the guide wire 60 to inflate the balloon 62 and further open the septal access opening created by the guide wire 60. In another embodiment, the balloon 62 may be connected to a catheter or sheath, e.g., the delivery catheter 52, and fluidly communicating with an external fluid reservoir as is well known in the art.

Additionally, or alternatively, to the balloon 62 disposed on the guide wire 60, the delivery catheter 52 may be used to deliver a tapered dilation member 64, which may be disposed on a corresponding catheter 66 as is well-known in the art, through the lumen of the delivery catheter 52, as shown in FIG. 9D. In such embodiments, the tapered member 64 may be used to further open the septal access opening created by the guide wire 60 and thereafter withdrawn through the delivery catheter 52. Next, the delivery catheter 52, with the prosthetic heart valve device 10, male engagement member 40, and at least a distal portion of the torque wire 50 received therein, is introduced into the vasculature, through the right atrium, and through the septal access opening until the distal end of the delivery catheter 52 and its lumen are positioned within the left atrium as in FIGS. 9D-9F.

Next, as shown in FIGS. 9G-9I, the torque wire 50 is pushed by the operator in the distal direction, thereby pushing the collapsed frame of the stent 12 out of the distal end of the lumen of the delivery catheter 52, whereby the collapsed frame of the stent 12 biasingly expands in the heart chamber of the left atrium. The torque wire 50 and/or the delivery catheter 52 are then manipulated by the operator to turn the delivery catheter 52, the torque wire 50, and/or the expanding prosthetic heart valve device 10 downward toward the mitral annulus for locating and seating. The skilled artisan will now appreciate that the various curvatures of the various embodiments of the stem region 46 of the male engagement member 40 may assist or enable such downward turning and/or other directional manipulation of the prosthetic heart valve device 10 during delivery of the same.

When the prosthetic heart valve device 10 is properly positioned in the exemplary left atrium, the torque wire 50 is then manipulated by the operator to disengage the male engagement member 40, as discussed with respect to FIG. 8A, from the stent cap 20. The torque wire 50 and the male engagement member 40 are then withdrawn proximally into the lumen of the delivery catheter 52. The delivery catheter 52 is then proximally withdrawn back through the septal access opening, as illustrated in FIGS. 9J-9L, and out of the patient, leaving the fully expanded and positioned prosthetic valve device 10 in place.

In some instances, it may be desirable to recapture the prosthetic valve device 10 after it has been delivered out of the lumen of the delivery catheter 52, either before or after the male engagement member 40 has been disconnected from the stent cap 20. For example, an operator delivering the prosthetic valve device 10 may determine that the device 10 is not approaching the mitral annulus at a desired angle, or that the device 10 is not properly located or seated at the mitral annulus. In instances in which the male engagement member 40 has not yet been disconnected from the stent cap 20 when this determination is made, recapture of the expanded device 10 may be achieved by distally pulling back the expanded device 10 with a distal proximal pulling of the torque wire 50 into the lumen of the delivery catheter 52 for controlled collapse of the device 10 therein. Alternatively, if the male engagement member 40 has been disconnected from the stent cap 20 when this determination is made, then the stent cap 20 may be reengaged with the male engagement member 40, as described above, for recapture. Reengagement of the stent cap 20 with the male engagement member 40 may be achieved using known visualization techniques; e.g., fluoroscopy, to guide recapture. Thus, in some embodiments, one or more portions of the male engagement member 40 and/or the stent cap 20 may include a radiopaque material.

In all embodiments, when the collapsed prosthetic heart valve device 10 is “loaded” within the lumen of the delivery catheter 52, it may be delivered via the delivery catheter 52 through the patient's vasculature to the heart chamber of interest using any acceptable access route and/or delivery technique, including but not limited to: transapical; transfemoral; transatrial; and transseptal delivery techniques, using the devices and systems and methods described above.

The skilled artisan will understand that the embodiments of the inventions described above may be used to improve implant loading of a prosthetic heart valve device into a delivery catheter, translation of the device through the lumen of a delivery catheter, controlled release of the device from the delivery catheter, positioning and locating of the expanding/expanded device in the subject heart chamber, repositioning and relocating of the device in the heart chamber, and/or recapturing and recollapsing of the device once expanded in the heart chamber.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

What is claimed is:

1. A loading, delivery, deployment and positioning system for an expandable and collapsible prosthetic heart valve device having a stent outer frame with a top and a bottom, wherein the bottom defines an outflow region therefrom, the prosthetic heart valve device biased to expand and adapted to collapse into the lumen of a delivery catheter, comprising:
a torque wire having a length that is longer than a length of the delivery catheter and adapted to translate and/or rotate within the delivery catheter lumen when a distal end of the torque wire is manipulated by an operator and wherein the torque wire comprises a threaded region at its distal end;
a stent cap non-rotationally attached at or near the top of the stent outer frame, the stent cap defining a channel and a pair of lateral locking grooves therethrough, wherein the channel is defined continuously with the lateral locking grooves;
a male engagement member comprising:
a threaded region at a proximal end, the threaded region adapted to threadingly engage the threaded region of the torque wire,
a stem region extending distally from the threaded region; and
left and right engagement handles extending laterally from a distal end of the stem region,
wherein the left and right engagement handles are adapted to detachably engage the stent cap.

2. The system of claim 1, wherein the stem region of the male engagement member is straight.

3. The system of claim 1, wherein the stem region of the male engagement member is curvilinear.

4. The system of claim 3, wherein the curvilinear stem region of the male engagement member comprises a single curved region.

5. The system of claim 3, wherein the curvilinear stem region of the male engagement member comprises more than one single curved region.

6. The system of claim 5, wherein each curved region of the stem region of the male engagement member comprises substantially similar curvature.

7. The system of claim 5, wherein each curved region of the stem region of the male engagement member comprises a curvature that differs from the curvature of at least one other curved region.

8. The system of claim 1, wherein the left and right engagement handles are adapted to detachably engage the stent cap when the stem region of the male engagement member is received within the channel such that the left and right engagement handles are positioned below the lateral locking grooves and within an interior of the stent frame.

9. The system of claim 1, wherein the prosthetic heart valve device comprises one of the group consisting of a prosthetic mitral valve, a prosthetic tricuspid valve and an aortic valve.

10. The system of claim 1, wherein the access route for the delivery catheter comprises at least one of the group consisting of: transapical, transfemoral, transatrial, and transseptal.

11. A method for loading, delivery, deployment and positioning a system for an expandable and collapsible prosthetic heart valve device, comprising:
providing the system of claim 1;
threadingly attaching the male engagement member to the torque wire;
removably attaching the male engagement member to the stent cap;
pulling the torque wire through the lumen of the delivery catheter in a proximal direction;
pulling the expanded prosthetic heart valve device into the distal end of the delivery catheter lumen and thereby collapsing the prosthetic heart valve device therein;
locating and loading the collapsed prosthetic heart valve device within the delivery catheter lumen;
accessing the patient's heart chamber with the distal end of the delivery catheter;
pushing with the torque wire the collapsed prosthetic heart valve device out of the distal end of the delivery catheter, whereby the prosthetic heart valve device biasingly expands;
rotating and/or otherwise turning the torque wire to direct and position the expanding prosthetic heart valve device within the heart chamber;
disconnecting the male engagement member from the stent cap;
withdrawing the torque wire and attached male engagement member into the lumen of the delivery catheter; and
withdrawing the delivery catheter from the patient's body.

12. The method of claim 11, wherein the stem region of the male engagement member is straight.

13. The method of claim 11, wherein the stem region of the male engagement member is curvilinear.

14. The method of claim 13, wherein the curvilinear stem region of the male engagement member comprises a single curved region.

15. The method of claim 13, wherein the curvilinear stem region of the male engagement member comprises more than one single curved region.

16. The method of claim 15, wherein each curved region of the stem region of the male engagement member comprises substantially similar curvature.

17. The method of claim 15, wherein each curved region of the stem region of the male engagement member comprises a curvature that differs from the curvature of at least one other curved region.

18. The method of claim 11, wherein the left and right engagement handles are adapted to detachably engage the stent cap when the stem region of the male engagement member is received within the channel such that the left and right engagement handles are positioned below the lateral locking grooves and within an interior of the stent frame.

19. The method of claim 11, wherein the prosthetic heart valve device comprises one of the group consisting of a prosthetic mitral valve, a prosthetic tricuspid valve and an aortic valve.

20. The method of claim 11, wherein the access route for the delivery catheter comprises at least one of the group consisting of: transapical, transfemoral, transatrial, and transseptal.
